(19) United States
(12) Patent Application Publication
(10) Pub. No.: US 2025/0134873 A1
(43) Pub. Date: May 1, 2025

(54) Pirfenidone Oral Suspensions

(71) Applicant: Dhruv Shah, Olathe, KS (US)

(72) Inventor: Dhruv Shah, Olathe, KS (US)

(73) Assignee: Catain Labs LLC, Olathe, KS (US)

(21) Appl. No.: 18/384,400

(22) Filed: Oct. 27, 2023

(51) Int. Cl. A61K 31/4418; A61K 9/00; A61K 47/12; A61K 47/26; A61K 47/38

(52) U.S. Cl. CPC A61K 31/4418; A61K 9/0053; A61K 47/12; A61K 47/26; A61K 47/38

(57) ABSTRACT

The present invention provides an aqueous pharmaceutical oral suspension of Pirfenidone at a concentration of 89 mg/ml, which would be useful to paediatric and geriatric patients because of the case of administration. In addition, one can easily titrate the dose in case required for individual patient dosage regimens.

### FIELD OF THE INVENTION

The present invention relates to pharmaceutical liquid suspension formulations of Pirfenidone suitable for oral administration, more particularly to liquid suspensions that remain homogeneous, easily re-dispersible and stable during storage.

### BACKGROUND OF THE INVENTION

### Description of Related Art

The most convenient and commonly employed oral drug formats are solids such as tablets and capsules. Older adults however have difficulty swallowing solid dosage formats, and in this case, the drug is given in liquid form, either as syrup or suspension.

Drugs are formulated as suspensions for different reasons, but the most common one is poor drug solubility. Suspensions may also be used to mask the poor taste resulting from the dissolved drug in solution. A suspension, however, unlike syrup in which the drug is fully dissolved, requires adequate shaking of the container to resuspend the drug uniformly before dosing. Difficult redispersion of the drug from a sediment, or in the worst case, from caking, will result in under-and overdosing. It is therefore desirable to produce a suspension that is able to re-disperse into a homogenous suspension with minimum shaking, maintain its homogeneity and be stable during storage.

Stoke's law defines the sedimentation rate of a sphere in a fluid and indicates that the rate of sedimentation can be reduced by minimizing the density difference between the suspended particles and the liquid phase, reducing the particle size, and increasing the viscosity of the liquid phase.

If the densities of the suspended particle and the liquid phase are the same, sedimentation will not occur. In practice, precise matching of the densities is not always possible. The drug density may be too high, or the amount of density increasing ingredients may be too great. Reducing the particle size is another way of slowing sedimentation. However, small particles tend to cake more severely because of the increased surface energy from the larger surface area, making redispersion much more difficult and sometimes impossible.

Small particle size is desirable for reasons other than slowing the rate of sedimentation. For drugs that are not very soluble, smaller particles generally dissolve faster due to the increase in the total surface area, which can in turn enhance bioavailability. Also, smaller drug particles are less likely to cause grittiness, which improves the palatability of the finished product. The most popular approach to slowing the sedimentation rate is by increasing the viscosity through the addition of a suspending agent. Excessive viscosity is undesirable, however, if it interferes with pouring and redispersal of settled particles.

The prior art has shown extensive use of combinations of suspending agents to promote redispersability. U.S. Pat. No. 4,975,465 discloses a tastemasked ibuprofen suspension comprising a suspending base of xanthan gum, microcrystalline cellulose, sodium carboxymethylcellulose and polysorbate 80. U.S. Pat. Nos. 5,272,137 and 5,409,907 teach the use of xanthan gum and microcrystalline cellulose to minimize sedimentation. U.S. Pat. Nos. 5,374,659 and 5,621,005 provide easily redispersable pharmaceutical suspensions using xanthan gum, pregelatinized starch and polyoxyethylene sorbitan monooleate. U.S. Pat. No. 5,658,919 discloses the use of xanthan gum, a mixture of microcrystalline cellulose and sodium carboxymethylcellulose, and an auxiliary suspending agent selected from hydroxyethylcellulose and a salt of carboxymethylcellulose to minimize sedimentation of paracetamol suspensions. U.S. Pat. No. 5,712,310 provides easily redispersable suspension base comprising a water-soluble modified starch, a water-soluble hydrocolloid polysaccharide, and a water-soluble wetting agent. U.S. Pat. No. 5,759,579 provides a liquid suspension base comprising xanthan gum and hydroxypropyl methylcellulose.

Pirfenidone is a synthetic pyridone drug. It is an antifibrotic agent with anti-inflammatory and antioxidant properties that is used to treat idiopathic pulmonary fibrosis (IPF), which is a chronic, progressive form of interstitial pneumonia. While its mechanism of action is not yet fully understood, pirfenidone is proposed to primarily regulate tumor necrosis factor (TNF) pathways and modulate cellular oxidation. The FDA first approved pirfenidone alongside nintedanib as one of the first drugs to treat IPF.

Pirfenidone was approved by FDA as Esbriet^{R} (Genentech) in both tablet and capsule dosage forms. The tablet dosage forms are available in strengths of 267 mg, 534 mg and 801 mg whereas the capsule dosage form is available in a single strength of 267 mg.

If one looks at the dosage regimen for pirfenidone it can be observed that the total recommended dosage per day irrespective of the dosage form is around 2403 mg per day which is significantly high. This implies that one has to consume almost 9 capsules/tablets (267 mg) or 3 tablets (801 mg). The 534 mg strength tablet has been discontinued by Genentech Inc.

The high recommended daily dose coupled with the physical size of the tablets especially for the 801 mg strength makes it very difficult to administer the daily dose to older population who have difficulties in swallowing such large dosage forms. For geriatric patients, an oral liquid suspension will overcome these practical difficulties of administration.

CN 101972230A relates to a pirfenidone suspension comprising pirfenidone, water soluble filler, disintegrating agent and a suspending agent. However, the patent application is silent as regards the particle size of pirfenidone, sedimentation, re-dispersibility, dissolution in different media and comparison of its bioequivalence with the FDA approved tablet/capsule formulation.

WO 2018234527 relates to aerosol delivery of pirfenidone by inhalation route. CN 111991393 and WO2021057599 relate to use of pirfenidone for treatment of rheumatoid arthritis.

### SUMMARY OF THE INVENTION

The present invention provides an aqueous pharmaceutical oral suspension of Pirfenidone at a concentration of 89 mg/ml, which would be useful to geriatric patients because of the ease of administration. In addition, one can easily titrate the dose in case it is needed to accommodate individual patient dosage regimens.

The present invention provides an aqueous pharmaceutical suspension for oral administration comprising pirfenidone with a particle size distribution of d₁₀<30 μm, d₅₀<100μm and d₉₀<300 μm; at least one anti-caking and suspending agent, at least one viscosity-building agent, along with preservative, sweetener, flavoring agent and solvent like sorbitol to yield a final suspension with a viscosity ranging from 75 to 200 cP, pH in the range of 5.5 to 6.5, redispersability of NLT 75% and a sedimentation of max 60% when allowed to stand for 24 hours. In addition, the oral suspension shows dissolution of NLT 80% Q in 20 minutes and the particle size remains constant during stability studies.

The aqueous suspension of this invention requires minimum shaking to get uniformly and homogenously dispersed, with a good content uniformity so that the amount of medication therefore remains uniform from dose to dose.

### DETAILED DESCRIPTION OF THE INVENTION

Aspects of the present specification disclose, in part, a pharmaceutical composition. As used herein, the term “pharmaceutically acceptable” means any molecular entity or composition that does not produce an adverse, allergic or other untoward or unwanted reaction when administered to an individual. As used herein, the term “pharmaceutically acceptable composition” is synonymous with “pharmaceutical composition” and means a therapeutically effective concentration of an active ingredient, such as, e.g., any of the therapeutic compounds disclosed herein. A pharmaceutical composition disclosed herein is useful for medical and veterinary applications. A pharmaceutical composition may be administered to an individual alone, or in combination with other supplementary active ingredients, agents, drugs or hormones.

A pharmaceutical composition disclosed herein may optionally include a pharmaceutically-acceptable carrier that facilitates processing of an active ingredient into pharmaceutically-acceptable compositions. As used herein, the term “pharmacologically-acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.” Such a carrier generally is mixed with an active compound or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, sorbitol solution, propylene glycol, glycerin and the like; suspending and viscosity building agents like hydroxypropylmethyl cellulose, carboxymethylcellulose sodium, microcrystalline cellulose, xanthan gum, alginates and the like; anti-caking agents such as stearates salts of calcium and magnesium, colloidal silicon dioxide, preservatives like sodium benzoate, methyl paraben, propyl paraben, potassium sorbate and the like, sweeteners like sodium saccharin, aspartame, sucralose, stevia and the like, flavoring agents like cherry flavor, guava flavor and the like based on the taste required or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7th ed. 1999); REMINGTON: THE SCIENCE AND PRACTICE OF PHARMACY (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4th edition 2003). These protocols are routine procedures and any modifications are well within the scope of one skilled in the art and from the teaching herein.

In one embodiment, a pharmaceutical composition disclosed herein comprises a therapeutic compound having anti-fibrotic activity and a pharmaceutically-acceptable adjuvant. In another embodiment, a pharmaceutical composition disclosed herein comprises a therapeutic compound having anti-fibrotic activity, a pharmaceutically-acceptable solvent, and a pharmaceutically-acceptable adjuvant. In aspects of this embodiment, a pharmaceutical composition disclosed herein may further comprise a pharmaceutically-acceptable stabilizing agent. In other aspects of this embodiment, a pharmaceutical composition disclosed herein may further comprise a pharmaceutically-acceptable carrier, a pharmaceutically-acceptable component, or both pharmaceutically-acceptable carrier and pharmaceutically-acceptable component.

In another embodiment, a pharmaceutical composition disclosed herein comprises the anti-fibrotic agent pirfenidone and a pharmaceutically acceptable carrier.

A pharmaceutical composition disclosed herein may comprise pirfenidone as the therapeutic agent in an amount of, e.g., less than about 10.0% (w/v), less than about 9.0% (w/v), less than about 8.0% (w/v), less than about 7.75% (w/v), less than about 7.50% (w/v), less than about 7.25% (w/v), less than about 7.00% (w/v), less than about 6.5% (w/v), less than about 6.25% (w/v), less than about 6.00% (w/v) less than about 5.50% (w/v) or less than about 5.0% (w/v). In other aspects of this embodiment, a pharmaceutical composition disclosed herein may comprise pirfenidone as the therapeutic agent in an amount in a range of, e.g., about 5.0% (w/v) to 6.00% (w/v), about 5.0% (w/v) to 6.25% (w/v), about 5.0% (w/v) to 6.50% (w/v), about5.0% (w/v) to 7.0% (w/v), about 5.0% (w/v) to 7.25% (w/v), about 5.0% (w/v) to 7.50% (w/v), about 5.0% (w/v) to 7.75% (w/v), about 5.0% (w/v) to 8.0% (w/v), about 5.0% (w/v) to 9.0% (w/v), or about 5.0% (w/v) to 10.0% (w/v).

A pharmaceutical composition disclosed herein may comprise a solvent in an amount of, e.g., less than about 95% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects of this embodiment, a pharmaceutical composition disclosed herein may comprise a solvent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

In another embodiment the pharmaceutical composition may comprise a solvent namely a polyhydric alcohol (also known as a polyol or sugar alcohol), an unsaturated aliphatic alcohol, an alicyclic alcohol, or a combination thereof. Examples of a monohydric alcohol include, without limitation, methanol, ethanol, propanol, butanol, pentanol, and 1-hexadecanol. Examples of a polyhydric alcohol include, without limitation, glycol, glycerol, arabitol, erythritol, xylitol, maltitol, sorbitol (gluctiol), mannitol, inositol, lactitol, galactitol (iditol), and isomalt. A preferred solvent of the invention is sorbitol.

A pharmaceutical composition disclosed herein may comprise a preservative in an amount of, e.g., less than about 5% (w/v), less than about 4% (w/v), less than about 3% (w/v), less than about 2% (w/v), less than about 1% (w/v), less than about 0.9% (w/v), less than about 0.8% (w/v), less than about 0.7% (w/v), less than about 0.6% (w/v), less than about 0.5% (w/v), less than about 0.4% (w/v), less than about 0.3% (w/v), less than about 0.2% (w/v), less than about 0.1% (w/v), less than about 0.05% (w/v) or less than about 0.01 (w/v). In other aspects of this embodiment, a pharmaceutical composition disclosed herein may comprise a preservative in an amount in a range of, e.g., about 0.01% (w/v) to 0.05% (w/v), about 0.01% (w/v) to 0.1% (w/v), about 0.01% (w/v) to 0.2% (w/v), about 0.01% (w/v) to 0.3% (w/v), about 0.01% (w/v) to 0.4% (w/v), about 0.01% (w/v) to 0.5% (w/v), about 0.01% (w/v) to 0.6% (w/v), about 0.01% (w/v) to 0.7% (w/v), about 0.01% (w/v) to 0.8%, about 0.01% (w/v) to 0.9% (w/v), about 0.01% (w/v) to 1.0% (w/v), about 0.01% (w/v) to 2.0% (w/v), about 0.01% (w/v) to 3.0% (w/v), about 0.01% (w/v) to 4.0% (w/v), or about 0.01% (w/v) to 5.0% (w/v).

In another embodiment the pharmaceutical composition may comprise a preservative namely sodium benzoate, methyl paraben, propyl paraben or potassium sorbate. A preferred preservative of the invention is sodium benzoate

A pharmaceutical composition disclosed herein may comprise a viscosity building agent in an amount of, e.g., less than about 3% (w/v), less than about 2.5% (w/v), less than about 2.25% (w/v), less than about 2% (w/v), less than about 1.5% (w/v), less than about 1.0% (w/v), less than about 0.75% (w/v), less than about 0.5% (w/v), less than about 0.25% (w/v) or less than about 0.1% (w/v), In other aspects of this embodiment, a pharmaceutical composition disclosed herein may comprise a viscosity building agent in an amount in a range of, e.g., about 0.1% (w/v) to 0.25% (w/v), about 0.1% (w/v) to 0.5% (w/v), about 0.1% (w/v) to 0.75% (w/v), about 0.1% (w/v) to 1.0% (w/v), about 0.1% (w/v) to 1.5% (w/v), about 0.1% (w/v) to 2.0% (w/v), about 0.1% (w/v) to 2.25% (w/v), about 0.1% (w/v) to 2.5% (w/v), or about 0.1% (w/v) to 3.0% (w/v).

In another embodiment the pharmaceutical composition may comprise a viscosity building agent namely hydroxypropylmethyl cellulose, carboxymethylcellulose sodium, microcrystalline cellulose, xanthan gum and alginate or its salts. A preferred viscosity building agent of the invention is carboxymethylcellulose sodium or Vivapur 591P^{R}. Vivapur 591P^{R} is a synergistic, co-processed composite consisting of microcrystalline cellulose (MCC) and sodium carboxymethylcellulose (Na-CMC). It is a unique suspending and emulsifying agent, which facilitates a wide range of liquid dosage forms. Due to its extraordinary stabilizing mechanism, it can be used with a broad variety of active pharmaceutical ingredients (APIs).

A pharmaceutical composition disclosed herein may comprise a stabilizer and anti-caking agent in an amount of, e.g., less than about 1% (w/v), less than about 0.5% (w/v), less than about 0.25% (w/v), less than about 0.2% (w/v), less than about 0.15% (w/v) or less than about 0.1% (w/v). In other aspects of this embodiment, a pharmaceutical composition disclosed herein may comprise a stabilizer and anti-caking agent in an amount in a range of, e.g., about 0.1% (w/v) to 0.15% (w/v), about 0.1% (w/v) to 0.2% (w/v), about 0.1% (w/v) to 0.25% (w/v), about 0.1% (w/v) to 0.5% (w/v) or about 0.1% (w/v) to 1.0% (w/v).

In another embodiment the pharmaceutical composition may comprise a stabilizer and anti-caking agent as stearates salts of calcium and magnesium, colloidal silicon dioxide. A preferred stabilizer and anti-caking agent of the invention is colloidal silicon dioxide.

In other aspects of this embodiment, a pharmaceutical composition disclosed herein may comprise a buffering agent in an amount in a range of, e.g., about 0.5% (w/v) to 1.0% (w/v), about 0.5% (w/v) to 1.5% (w/v), about 0.5% (w/v) to 2.0% (w/v), about 0.5 (w/v) to 2.25% (w/v) or about 0.5% (w/v) to 2.5% (w/v).

In another embodiment the pharmaceutical composition may comprise a buffering agent asuch as citrate, phosphate, aminoacids like histidine, gluconodeltalactone to adjust pH in the range of 5.5 to 6.5.

A pharmaceutical composition disclosed herein may comprise a co-processed excipient of silicon dioxide and microcrystalline cellulose (Prosolv SMCC 50^{R}) in an amount of, e.g., less than about 2.5% (w/v), less than about 2.25% (w/v), less than about 2.0% (w/v), less than about 1.5% (w/v), less than about 1.0% (w/v) or less than about 0.5% (w/v).

In other aspects of this embodiment, a pharmaceutical composition disclosed herein may comprise a suspending and anti-caking agent in an amount in a range of, e.g., about 0.5% (w/v) to 1.0% (w/v), about 0.5% (w/v) to 1.5% (w/v), about 0.5% (w/v) to 2.0% (w/v), about 0.5% (w/v) to 2.25% (w/v) or about 0.5% (w/v) to 2.5% (w/v).

In another embodiment the pharmaceutical composition may comprise a suspending and anti-caking agent as a co-processed excipient of silicon dioxide and microcrystalline. A preferred suspending and anti-caking agent of the invention is Prosolv SMCC 50^{R}.

A pharmaceutical composition disclosed herein may comprise a flavouring and optionally a coloring agent in a concentration range of 0.1 to 2% w/v and 0.0001 to 0.002% w/v respectively to impart a palatable and organoleptic properties to the oral suspension. Preferred flavoring agents include cherry flavor and F D&C Red 40 a suitable coloring agent.

The invention will now be described with respect to the following examples:

All the examples described herein used a particle size of pirfenidone with a particle size distribution of d₁₀<30 μm, d₅₀<100 μm and d₉₀<300 μm;

### Example 1

| | | | Quantity per |
|---|---|---|---|
| Sr. No | % W/W | Material/Component | mL (mg) |
| 1 | 6.50 | Pirfenidone | 89.000 |
| 2 | 92.40 | Sorbitol Solution 70% | 1264.956 |
| 3 | 0.10 | Sodium Benzoate | 1.369 |
| 4 | 1.00 | Cherry Flavor | 13.690 |
| 5 | 0.00 | FD&C Red No 40 | 0.014 |
| Total | 100.00 | | 1369.029 |

### Observation

1. Sodium Benzoate is insoluble on Sorbitol Solution 70%.
2. Sodium Benzoate dissolved in Cherry Flavor after mixing for 15-20 mins at high rpm (400-700 rpm).
3. Addition of Pirfenidone in Sorbitol Solution 70% did not create any agglomeration and was mixed consistently at medium to high rpm (400-700 rpm) resulting in consistent looking suspension.
4. Upon leaving the suspension overnight, the active does not sediment. In fact, the active came on the surface of sorbitol solution 70% due to high density of sorbitol solution.
5. Upon shaking, the active disperse evenly resulting in suspension.

### Example 2

| | | | Quantity per |
|---|---|---|---|
| Sr. No | % W/W | Material/Component | mL (mg) |
| 1 | 7.416700 | Pirfenidone | 89.000 |
| 2 | 9.272500 | Sorbitol Solution 70% | 111.270 |
| 3 | 1.140000 | Vivapur 591 P | 13.680 |
| 4 | 0.500000 | Citric Acid Anhydrous | 6.000 |
| 5 | 0.100000 | Sodium Benzoate | 1.200 |
| 6 | 0.187500 | Colloidal Silicon | 2.250 |
| | | Dioxide | |
| 7 | 80.382300 | Purified Water | 964.588 |
| 8 | 1.000000 | Cherry Flavor | 12.000 |
| 9 | 0.001000 | FD&C Red No 40 | 0.012 |
| Total | 100.000000 | | 1200.000 |

### Observation

1. The density of the suspension change, resulting in sedimentation of the API.
2. The addition of anticaking agent did help easy dispersion of the API upon shaking.

### Example 3

| | | | Quantity per |
|---|---|---|---|
| Sr. No | % W/W | Material/Component | mL (mg) |
| 1 | 7.126500 | Pirfenidone | 89.004 |
| 2 | 90.928700 | Sorbitol Solution 70% | 1135.619 |
| 3 | 0.912000 | Vivapur 591 P | 11.390 |
| 4 | 0.152000 | Colloidal Silicon | 1.898 |
| | | Dioxide | |
| 5 | 0.080000 | Sodium Benzoate | 0.999 |
| 6 | 0.800000 | Cherry Flavor | 9.991 |
| 7 | 0.000800 | FD&C Red No 40 | 0.010 |
| Total | 100.000000 | | 1248.911 |

### Observation

1. The density of the suspension increased resulting in API coming to the surface of suspension. Re-dispersibility was not an issue and was easily achieved by minimal shaking.

### Example 4

| | | | Quantity per |
|---|---|---|---|
| Sr. No | % W/W | Material/Component | mL (mg) |
| 1 | 7.691700 | Pirfenidone | 89.001 |
| 2 | 61.890000 | Sorbitol Solution 70% | 716.134 |
| 3 | 2.700000 | Vivapur 591 P | 31.242 |
| 4 | 0.152000 | Colloidal Silicon | 1.759 |
| | | Dioxide | |
| 5 | 26.685500 | Purified Water | 308.780 |
| 6 | 0.080000 | Sodium Benzoate | 0.926 |
| 7 | 0.800000 | Cherry Flavor | 9.257 |
| 8 | 0.000800 | FD&C Red No 40 | 0.009 |
| Total | 100.000000 | | 1157.107 |

### Observation

1. The particles in the suspension did not sediment or came on the surface. However, the viscosity of suspension appeared very high.
2. The suspension did taste a little bitter.

### Example 5

| | | | Quantity per |
|---|---|---|---|
| Sr. No | % W/W | Material/Component | mL (mg) |
| 1 | 7.691700 | Pirfenidone | 89.000 |
| 2 | 61.890000 | Sorbitol Solution 70% | 716.126 |
| 3 | 1.800000 | Vivapur 591 P | 20.828 |
| 4 | 0.152000 | Colloidal Silicon | 1.759 |
| | | Dioxide | |
| 5 | 27.335500 | Purified Water | 316.298 |
| 6 | 0.250000 | Sodium Saccharin | 2.893 |
| | | Dihydrate | |
| 7 | 0.080000 | Sodium Benzoate | 0.926 |
| 8 | 0.800000 | Cherry Flavor | 9.257 |
| 9 | 0.000800 | FD&C Red No 40 | 0.009 |
| Total | 100.000000 | | 1157.095 |

### Observation

1. The appearance of suspension improved very much. However, upon leaving the suspension overnight, it did sediment. Re-dispersion was not an issue and was achieved upon minimum shaking.
2. Addition of Sodium Saccharin Dihydrate improved the taste of suspension.

### Example 5

| | | | Quantity per |
|---|---|---|---|
| Sr. No. | % W/W | Material/Component | mL (mg) |
| 1 | 7.691700 | Pirfenidone | 89.000 |
| 2 | 61.890000 | Sorbitol Solution 70% | 716.126 |
| 3 | 2.200000 | Vivapur 591 P | 25.456 |
| 4 | 0.152000 | Colloidal Silicon | 1.759 |
| | | Dioxide | |
| 5 | 26.935500 | Purified Water | 311.669 |
| 6 | 0.250000 | Sodium Saccharin | 2.893 |
| | | Dihydrate | |
| 7 | 0.080000 | Sodium Benzoate | 0.926 |
| 8 | 0.800000 | Cherry Flavor | 9.257 |
| 9 | 0.000800 | FD&C Red No 40 | 0.009 |
| Total | 100.000000 | | 1157.095 |

### Observation

1. The appearance of suspension improved. The suspension hardly sediment upon leaving overnight. Re-dispersion was easily achieved on minimum shaking.
2. Specific gravity of suspension was found to be 1.218 g/mL.

Due to supply chain restriction for Vivapur 591, it was decided to substitute Vivapur 591P and Colloidal Silicon Dioxide with Carboxymethylcellulose Sodium, Medium Viscosity (from Spectrum Chemical Mfg Corp) and Prosolv SMCC 50 (from JRS). The formulation was adjusted to keep the percentage of each excipient in similar range as finalized during formulation development.

### Example 6

| | | | Quantity per |
|---|---|---|---|
| Sr. No | % W/W | Material/Component | mL (mg) |
| 1 | 7.30 | Pirfenidone | 89.001 |
| 2 | 27.96 | Purified Water | 340.762 |
| 3 | 0.25 | Carboxymethylcellulose | 3.000 |
| | | Sodium Medium Viscosity | |
| 4 | 0.08 | Sodium Benzoate | 0.926 |
| 5 | 0.24 | Sodium Saccharin dihydrate | 2.893 |
| 6 | 2.10 | Prosolv SMCC 50 | 25.600 |
| 7 | 54.95 | Sorbitol Solution 70% | 669.800 |
| | | Solution | |
| 8 | 0.98 | Cherry Flavor | 11.900 |
| 9 | 6.15 | Sorbitol Solution 70% | 75.000 |
| | | Solution | |
| Total | 100.00 | | 1218.928 |

### Observation

1. The suspension met all the acceptable test criteria for appearance, pH, specific gravity, taste and aroma.
2. The suspension did settle on leaving for 24 hours but re-dispersion was easily achieved on minimum shaking.

### Example 7

| | | | Quantity per |
|---|---|---|---|
| Sr. No | % W/W | Material/Component | mL (mg) |
| 1 | 7.69 | Pirfenidone | 89.001 |
| 2 | 62.15 | Sorbitol Solution 70% | 719.142 |
| 3 | 2.19 | Vivapur 591 P | 25.387 |
| 4 | 0.15 | Colloidal Silicon | 1.759 |
| | | Dioxide | |
| 5 | 26.68 | Purified Water | 308.762 |
| 6 | 0.25 | Sodium Saccharin | 2.893 |
| | | dihydrate | |
| 7 | 0.08 | Sodium Benzoate | 0.926 |
| 8 | 0.80 | Cherry Flavor | 9.257 |
| 9 | 0.001 | FD&C Red No 40 | 0.009 |
| Total | 100.00 | | 1157.136 |

### Formulation Process

1. In a container, following materials are added in order.
   a. Purified Water
   b. Vivapur 591 P
   c. Sorbitol Solution
   d. Sodium Benzoate
   e. Sodium Saccharin Dihydrate
   f. Colloidal Silicon Dioxide
   g. Cherry Flavor
   h. FD&C Red 40
   i. Pirfenidone
2. The suspension was under continuous mixing with rpm 400-700.

### Observation

1. Upon addition of Vivapur 591P to purified water, it forms almost gel/cream like texture, resulting in difficulty of mixing. Hence, Sorbitol solution 70% was added immediately to decrease the viscosity of suspension.
2. The suspension formed at the end was comparatively viscous with viscosity of 775 mPa*s measured using rotor 2 at 30 rpm.
3. The pH of the suspension was found to be 6.0
4. The assay of the suspension was about 106% (n=3).
5. The dissolution of suspension was less than 80% in 30 mins.

### Example 8

| | | | Quantity per |
|---|---|---|---|
| Sr No | % W/W | Material/Component | mL (mg) |
| 1 | 7.30 | Pirfenidone | 89.001 |
| 2 | 61.28 | Sorbitol Solution 70% | 747.000 |
| 3 | 2.16 | Vivapur 591 P | 26.387 |
| 4 | 0.14 | Colloidal Silicon | 1.759 |
| | | Dioxide | |
| 5 | 25.33 | Purified Water | 308.762 |
| 6 | 0.24 | Sodium Saccharin | 2.893 |
| | | dihydrate | |
| 7 | 0.08 | Sodium Benzoate | 0.926 |
| 8 | 0.80 | Cherry Flavor | 9.700 |
| 9 | 2.67 | Purified Water | 32.500 |
| Total | 100.00 | | 1218.928 |

### Formulation Process

1. In a container, following materials are added in order.
   a. Purified Water
   b. Sodium benzoate
   c. Sodium Saccharin Dihydrate
   d. Vivapur 591 P
   e. Colloidal Silicon Dioxide
   f. Cherry Flavor
   g. FD&C Red 40
   h. Sorbitol Solution
   i. Pirfenidone
2. The suspension was under continuous mixing with rpm 400-700.

### Observation

1. Upon addition of Vivapur 591P to a mixture purified water+sodium benzoate+sodium saccharin dihydrate, there was no viscosity observed for the suspension.
2. The suspension formed at the end had very low viscosity (less than 50 mPa*s).
3. The pH of the suspension was found to be 6.0
4. The assay of the suspension was about 100% (n=3).
5. The dissolution of suspension was more than 80% in 20 mins.

### Example 9

| | | | Quantity per |
|---|---|---|---|
| Sr. No | % W/W | Material/Component | mL (mg) |
| 1 | 7.30 | Pirfenidone | 89.001 |
| 2 | 61.10 | Sorbitol Solution 70% | 744.800 |
| 3 | 2.21 | Vivapur 591 P | 26.887 |
| 4 | 0.14 | Colloidal Silicon | 1.759 |
| | | Dioxide | |
| 5 | 25.33 | Purified Water | 308.762 |
| 6 | 0.24 | Sodium Saccharin | 2.893 |
| | | dihydrate | |
| 7 | 0.08 | Sodium Benzoate | 0.926 |
| 8 | 0.98 | Cherry Flavor | 11.900 |
| 9 | 2.63 | Purified Water | 32.000 |
| Total | 100.00 | | 1218.928 |

### Formulation Process

1. In a container, following materials are added in order.
   a. Purified Water
   b. Sodium benzoate
   c. Vivapur 591P
   d. Sodium Saccharin Dihydrate
   c. Colloidal Silicon Dioxide
   f. Cherry Flavor
   g. FD&C Red 40
   h. Sorbitol Solution
   i. Pirfenidone
2. The suspension was under continuous mixing with rpm 400-700.

### Observation

1. Upon addition of Vivapur 591P to a mixture purified water+sodium benzoate, the viscosity of suspension was found to be adequate.
2. The suspension formed at the end had acceptable viscosity level (between 100 to 500 mPa*s)
3. The pH of the suspension was found to be 6.0
4. The assay of the suspension was about 100% (n=3).
5. The dissolution of suspension was more than 80% in 30 mins. The dissolution seemed little slower compared to RLD and specification requirement of NLT 80% in 20 mins.

### Example 10

| | | | Quantity per |
|---|---|---|---|
| Sr. No | % W/W | Material/Component | mL (mg) |
| 1 | 7.30 | Pirfenidone | 89.001 |
| 2 | 27.96 | Purified Water | 340.762 |
| 3 | 0.25 | Carboxymethylcellulose | 3.000 |
| | | Sodium Medium Viscosity | |
| 4 | 0.08 | Sodium Benzoate | 0.926 |
| 5 | 0.24 | Sodium Saccharin dihydrate | 2.893 |
| 6 | 2.10 | Prosolv SMCC 50 | 25.600 |
| 7 | 54.95 | Sorbitol Solution 70% | 669.800 |
| | | Solution | |
| 8 | 0.98 | Cherry Flavor | 11.900 |
| 9 | 6.15 | Sorbitol Solution 70% | 75.000 |
| | | Solution | |
| Total | 100.00 | | 1218.928 |

### Formulation Process

1. In a container, following materials are added in order.
   a. Purified Water
   b. Carboxymethylcellulose Sodium, Medium Viscosity
   c. Sodium benzoate
   d. Sodium Saccharin Dihydrate
   e. Prosolv SMCC 50
   f. Sorbitol Solution 70%
   g. Pirfenidone
   h. Sorbitol Solution 70%
   i. Cherry Flavor
2. The suspension was under continuous mixing with rpm 300-600.

### Observation

1. Upon addition of Carboxymethylcellulose Sodium, Medium Viscosity to purified water, the viscosity of suspension was found to be adequate.
2. The suspension formed at the end had acceptable viscosity level (between 750 to 250 mPa*s)
3. The pH of the suspension was found to be 6.0
4. The assay of the suspension was about 100% (n=3).
5. The dissolution of suspension was more than 80% in 20 mins.

### Example 11

| | | Quantity per |
|---|---|---|
| % w/w | Material/Component | mL (mg) |
| 7.30 | Pirfenidone | 89.00 |
| 27.96 | Purified Water | 340.76 |
| 0.25 | Carboxymethylcellulose Sodium | 3.00 |
| | (Aqualon ™ CMC 7 MF PH) | |
| 0.08 | Sodium Benzoate | 0.93 |
| 0.24 | Sodium Saccharin Dihydrate | 2.89 |
| 2.10 | Silicified Microcrystalline | 25.60 |
| | Cellulose (Prosolv SMCC 50) | |
| 61.10 | Sorbitol Solution | 744.80 |
| | (Neosorb 70/20) | |
| 0.98 | Cherry Flavor 825.611 | 11.90 |
| 100.01 | | 1218.88 |

### Final Manufacturing Process of Drug Product

1. Main tank mixing
   a. Addition of Purified Water and begin mixing.
   b. Addition of Aqualon™ CMC 7MF PH and mix until completely dissolved.
   c. Addition of Sodium Benzoate and mix until completely dissolved.
   d. Addition of Sodium Saccharin Dihydrate and mix until completely dissolved.
   e. Addition of Prosolv SMCC 50 and mix until completely dispersed.
   f. Addition of about 90% of Neosorb 70/20 Sorbitol and mix until completely dispersed.
   g. Addition of Pirfenidone and mix until completely dispersed.
   h. Rinse the container of Pirfenidone with remaining 10% Neosorb 70/20 Sorbitol and add and mix into the suspension until completely dispersed.
   i. Addition of Nat-Art Cherry Flavor #825.6111 and mix until completely dispersed.
   j. Fill the solution in 16 oz PET Amber bottle with 28 mm child-resistant closure and labeled.

### In-Vitro Multi-Media Dissolution

Dissolution studies were conducted on Example 11 which is the final formulation in four different media namely 0.1N HCl (pH 1.0), acetate buffer (pH 4.5), phosphate buffer (pH 6.0) and water. The results are tabulated below:

TABLE 1
Multi-media dissolution of the Example 11 formulation

| | Mean percentage of pirfenidone dissolved | | | |
|---|---|---|---|---|
| Time in | 0.1N HCl | Acetate buffer | Phosphate buffer | |
| minutes | pH 1.0 | pH 4.5 | pH 6.0 | Water |
| 10 | 68.3 | 58.5 | 66.1 | 62.3 |
| 20 | 95.2 | 89.4 | 93.5 | 87.2 |
| 30 | 96.3 | 92.5 | 96.3 | 92.5 |
| 40 | 96.7 | 92.9 | 96.9 | 95.8 |
| 60 | 97.0 | 93.4 | 97.4 | 97.8 |

Observation: It was observed that NLT 80% Q dissolution was obtained for the Example 11 formulation in all media.

1. An oral liquid pirfenidone composition comprising from about 5 to about 10 percent by weight pirfenidone with a particle size distribution of d₁₀<30 μm, d₅₀<100 μm and d₉₀<300 μm; at least one anti-caking and suspending agent, at least one viscosity-building agent, along with preservative, sweetener, flavoring agent and solvent to yield a final suspension with a viscosity ranging from 75 to 200 cP, pH in the range of 5.5 to 6.5, redispersability of NLT 75%, with a dissolution of NLT 80% Q in 20 minutes and stable during accelerated stability.

2. A composition of claim 1, wherein the anti-caking and suspending agent is silicified microcrystalline cellulose

3. A composition of claim 1, wherein the viscosity building agent is carboxymethylcellulose or its salts.

4. A composition of claim 1, wherein the preservative is sodium benzoate.

5. A composition of claim 1, wherein the sweetener is saccharin sodium.

6. A composition of claim 1, wherein the solvent is sorbitol.

7. A composition of claim 1, wherein the flavoring agent is Cherry Flavor.

8. A composition of claim 1, with a pH between 5 and 6.5.

9. A composition of claim 1, wherein multi-media dissolution in 0.IN HCl, acetate buffer, phosphate buffer and water releases NLT 80% Q of pirfenidone in less than 20 minutes.

10. A composition of claim 1, which is stable at normal and accelerated stability storage conditions.
